(19) United States
(12) Patent Application Publication
(10) Pub. No.: US 2006/0046970 A1
(43) Pub. Date: Mar. 2, 2006

(54) Topical otic compositions and methods of topical treatment of prevention of otic infections

(75) Inventors: Lyle M. Bowman, Pleasanton, CA (US); Santosh Kumar Chandrasekaran, Moraga, CA (US); Erwin C. Si, Alameda, CA (US)

Correspondence Address: SUGHRUE MION, PLLC, 2100 PENNSYLVANIA AVENUE, N.W., SUITE 800, WASHINGTON, DC, 20037, US

(73) Assignee: INSITE VISION INCORPORATED

(21) Appl. No.: 10/929,546

(22) Filed: Aug. 31, 2004

(51) Int. Cl. A61K 31/7048; A61K 31/195; A61K 31/192

(52) U.S. Cl. 514/28; 514/569; 514/570; 514/567; 514/171

(57) ABSTRACT

A topical otic composition containing an azalide antibiotic. A topical otic composition containing an azalide antibiotic and an medicament. A topical otic composition containing an azalide antibiotic and a polymer suspending agent. And methods for treating or preventing infections in the ear using azalide antibiotic compositions.

### BACKGROUND OF THE INVENTION

1. Field of the Invention

The present invention relates to topical otic compositions containing an azalide antibiotic and to the use of azalide antibiotics in methods for treating and/or preventing infections in the ear.

2. Description of the Related Arts

Azalide antibiotic formulations are known for the use in the treatment of ophthalmic infections. See U.S. Pat. Nos. 6,239,113 B1 and 6,569,443 B1 and U.S. Patent Application Publications 2003/0206956 A1 and 2003/0143259 A1 to InSite Vision, Inc.

Otic infections may be treated by local injection, systemic administration, or topical application of an antibiotic. However, treating infections in otic tissues remains challenging and/or problematic because of the difficulty in delivering an antibiotic to the affected tissue.

The simple and direct approach of topically applying the antibiotic to the ear has several benefits, including the avoidance of side effects and the reduced chance of developing resistant strains of bacteria as compared to systemic administration. However, for a variety of reasons, many antibiotics are not suitable for topical application to the ear.

Another concern is that the antibiotic will be toxic to the ear. A toxic response includes redness and swelling and/or discharge. Toxicity is especially problematic for topical administration because it is a concentration dependent phenomenon. While a drug may be non-toxic at the minimum effective concentration, an increase in concentration such as associated with topical administration may well induce a toxic response. The fact that oral or systemic administration shows the drug to be compatible with otic tissue does not predict or address the toxicity issue associated with topical administration.

A further unsuitability of topical antibiotics is the practicality of topical administration by the patient. Assuming that sufficiently high concentrations of the antibiotic can be used to achieve an effective dose within the target tissue without a toxic response, the application may nonetheless be irritating. An irritation response includes temporary burning or stinging and/or causing inflammation. The patient may simply be resistant to complying with the dosage regimen because of the irritation. By failing to comply with the dosing regimen, the treatment efficacy is reduced or eliminated.

Examples of antibiotics that are used in otic topical administration include fluoroquinolones, aminoglycosides, macrolides and sulfonamides. However, the dosing of the known topical antibiotics is usually an extensive and inconvenient regimen. Such an extensive dosing regimen is inconvenient and obtaining patient compliance can be difficult. The greater the non-compliance with the regimen, the less effective the treatment.

Quinolone antibiotics, such as ciprofloxacin, have been previously utilized to treat otic infections. However, despite the general efficacy of quinolone therapies currently available, there is a need for improved compositions and methods of treatment based on the use of antibiotics that are more effective than existing antibiotics against key otic pathogens, particularly bacterial infections, and less prone to the development of resistance by those pathogens.

The use of oral antibiotics to treat otic infections in children has limited efficacy, and creates a serious risk of pathogen resistance to the orally administered antibiotics.

Thus, there continues to be a need for antibiotics that are effective in the topical treatment of the ear. It is desirable to provide a topical formulation that is effective against a broad spectrum of bacteria and that can be administered in an easy, effective regimen.

### SUMMARY OF THE INVENTION

Applicants have discovered that azalide antibiotics are suitable for topical administration to the ear for the treatment and/or prevention of otic infections. The present invention relates to topical otic compositions containing an azalide antibiotic and methods of using such topical otic compositions for treating the ear. The treatment comprises topically applying an azalide antibiotic to an ear in an amount effective to treat or prevent infection in a tissue of the ear.

The present invention includes the use of a topical otic composition containing at least one azalide antibiotic to treat otic infections, as well as the use of a topical otic composition prior to surgery to sterilize the surgical field and prophylactically following surgery or other trauma to otic tissue to minimize the risk of infection. The topical otic composition of the present invention may also be administered to the affected tissues during otic surgical procedures to prevent or alleviate post-surgical infection. As utilized herein, the terms “treat”, “treating” and derivations thereof are intended to include both treatments of existing infections and treatments to prevent or reduce the risk of infections.

The topical otic composition of the present invention is formulated for topical application to otic tissue. The topical otic composition is preferably sterile, and has physical properties (e.g., osmolality and pH) that are specially suited for application to otic tissue, including tissues that have been compromised as the result of preexisting disease, trauma, surgery or other physical conditions. The concentration of the antibiotic(s) in the topical otic composition of the present invention will vary depending on the intended use of the composition (e.g., treatment of existing infections or prevention of post-surgical infections), and the antimicrobial activity of the specific antibiotic(s) selected.

The topical otic composition may be administered to the affected otic tissue by topically applying a sterile solution or suspension, or a comparable amount of an ointment, gel or other solid or semisolid composition. The topical otic composition may also be formulated as an irrigating solution that is applied to the affected otic tissue during cleaning or surgical procedures.

A preferred form of the present invention involves forming or supplying a depot of the azalide antibiotic in contact with the ear for a sufficient length of time to allow a minimum inhibitory concentration (MIC) of the azalide antibiotic to diffuse into the cells of the targeted ear tissue(s). Once the MIC threshold has been surpassed, a therapeutically effective concentration of the azalide antibiotic will remain in the tissue(s) for a considerable period of time due to its long half-life. Accordingly, an advantage of certain preferred forms of the present invention is a simplified dosing regimen. For example, one or two topical applications may provide a sufficient tissue concentration that an inhibitory concentration remains resident in the infected tissue for several days. Thus, a complete treatment regimen may involve only one or two topical applications.

The present invention relates to a topical otic composition containing an azalide antibiotic. The present invention does not involve methods of systemic treatment or a systemic composition. In one embodiment, the topical otic composition may be a sustained release composition comprised of an aqueous suspension of the azalide antibiotic, a polymer suspending agent and a wetting agent. In one embodiment, the otic composition may be a sustained release composition comprised of an aqueous suspension of the azalide antibiotic and a polymer suspending agent.

### DETAILED DESCRIPTION OF THE INVENTION

Azalides are a known subclass of macrolide antibiotics. Occasionally, the literature has also referred to these compounds as azolides, and the two spellings should be taken as having the same meaning.

The azalide antibiotics used in the present invention are commercially available or readily obtained by a worker skilled in the art through known reactions techniques. The azalide antibiotics can be formed from erythromycin A, a naturally occurring compound formed during the culturing of a strain of Streptomyces erythreus. However, it is not required that the azalide antibiotic actually be formed from erythromycin.

An “azalide antibiotic” may be a derivitized erythromycin A structure having a nitrogen atom inserted into the lactone ring. Additional variations from the erythromycin structure are also embraced within the term “azalide antibiotic.” Such additional variations include the conversion of a hydroxyl group to an alkoxy group, especially methoxy (so-called “O-methylated” forms), for example at the 6 and/or 12 position. Such compounds are described in U.S. Pat. No. 5,250,518, the entire contents of which are incorporated herein by reference. Other variations relate to derivatives of the sugar moieties, for example, 3″ desmethoxy derivatives and the formation of oxo or oxime groups on the sugar ring such as at the 4″ position as described in U.S. Pat. No. 5,441,939, the entire contents of which are incorporated herein by reference. This patent also teaches that the adjacent hydroxyl groups at the 11 and 12 position of the lactone ring can be replaced with a single carbonate or thiocarbonate group. In short, an azalide antibiotic for purposes of the present invention is any derivative of the erythromycin structure that contains a 15-member lactone ring having a ring nitrogen, preferably at the 9 position, and a sugar group attached via a glycosidic bond to the lactone ring at the 5 position and at the 3 position, and which still exhibits bacteriostatic or bactericidal activity.

Preferred azalide antibiotics are represented by formula (1) and pharmaceutically acceptable salts thereof.

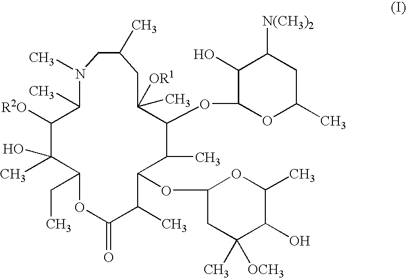

R¹ and R² each independently represent a hydrogen atom or a methyl group. Preferably at least one of R¹ and R² is a hydrogen atom. Azithromycin, the common name for N-methyl-11-aza-10-deoxo-10-dihydroerythromycin, corresponds to the compound of formula (I) where both R¹ and R² are a hydrogen atom. Azithromycin is disclosed in U.S. Pat. Nos. 4,474,768 and 4,517,359, the entire contents of each patent being incorporated herein by reference, and is the most preferred azalide antibiotic. One form of azalide is the dihydride form of azithromycin (azithromycin dihydrate).

Azithromycin has been used as an oral antibiotic and is sold worldwide under the brand name Zithromax® by Pfizer Inc. Azithromycin is a broad spectrum antibiotic that is generally more effective in vitro than erythromycin. Moreover, because azithromycin is an azalide and thus has a ring nitrogen atom, it exhibits improved acid-stability, half-life, and cellular uptake in comparison to erythromycin. The high uptake and retention of azithromycin into cells, including phagocytic blood cells, allows the systemically administered azithromycin to be nonetheless preferentially delivered to the site of the infection. The mechanism is believed to be as follows: The ingested azithromycin is absorbed through the intestine into the blood stream from which it enters most cells of the body including, inter alia, the white blood cells. In response to an infection within the body, white blood cells, including those containing azithromycin, are attracted to the infectious site. When the white blood cells die, the azithromycin is released. As more and more white blood cells arrive at the infectious site and die, the concentration of azithromycin in the surrounding tissue increases, eventually surpassing the MIC. Once at the infectious site, the azithromycin remains in the tissue for a prolonged period of time, due to its long half-life, such that an effective concentration of azithromycin is present at the infected site for many days after cessation of administration.

Although azithromycin can reach many of the tissues and fluids of the ear by oral administration, it has now been discovered that azalide antibiotics in general and azithromycin in particular are amenable to topical administration in or on the ear.

The ear is susceptible to bacterial and parasitic infections arising from both traumatic and non-traumatic related events. Infections are a concern after otic surgery and precautions are correspondingly taken to prevent the onset of infection. However, even without the invasive trauma of a surgical procedure, infections in the ear and otic tissues often occur.

Examples of otic conditions that may be treated with the compositions of the present invention include otitis externa and otitis media, ear inflammation, ear infections and ear trauma. Examples of bacteria believed to act as pathogens in acute otitis externa infections include “corneforms” or “idphtheroids”. They have previously been identified as being present both in healthy ears and in ears afflicted with acute otitis externa infections.

With respect to the treatment of otitis media, the compositions of the present invention may be useful in cases where the tympanic membrane has ruptured or tympanostomy tubes have been implanted. The compositions may also be used to treat infections associated with otic surgical procedures, such as tympanostomy, or to prevent such infections. The compositions and methods of the present invention may be useful in the treatment of acute infections of the external ear canal, which are commonly referred to as “acute otitis externa” or “AOE”. The antibiotics utilized in the present invention have a high level of antimicrobial activity against otic pathogens, and therefore may be useful in the treatment of acute otitis externa infections involving these pathogens.

The azalide antibiotic can be supplied to otic tissue in a variety of ways, including as an aqueous otic solution or suspension, as an otic oil, oil solution, and as an otic insert but the application is not limited thereto. Any technique, and topical otic composition containing a dosage form that supplies an azalide antibiotic to otic tissues is included within the notion of “topically applying.” Although the external surface of the ear is typically the ear canal.

The amount of azalide antibiotic topically supplied is effective to treat or prevent infection in a tissue of the ear. This means that the conditions of application result in a retarding or suppression of the infection. Typically at least about MIC₅₀ for the targeted bacteria or parasite is delivered to the otic tissue by the topical application of an effective amount. More concretely, the concentration within the otic tissue is desired to be at least about 0.25 μg/g, preferably at least 1 μg/g, and more preferably at least 10 μg/g. The amount of azalide actually supplied to the otic tissue surface will almost always be much higher than the tissue concentration. This reflects the penetration hold up of the azalide antibiotic by the outer tissue layers of the ear and that penetration is to some extent concentration driven. Thus, supplying greater amounts to the exterior will drive more antibiotic into the tissues.

Where a series of applications are used in the dosing regimen, it is possible that one or more of the earlier applications will not achieve an effective concentration in the otic tissue, but that a later application in the regimen will achieve an effective concentration. This is contemplated as being within the scope of topically applying an azalide antibiotic in an effective amount. However, generally a single application, such as consisting of one or two drops, provides a therapeutically effective concentration (e.g. one that retards or suppresses the infection) of the azalide antibiotic within a tissue of the ear. Indeed, although dependent on the amount and form of the otic composition, a single application will typically provide a therapeutically effective amount of the azalide antibiotic within a tissue of the ear for at least 12, preferably 18, and more preferably at least 24 hours

The topical application of an azalide antibiotic may be used to treat or prevent a variety of conditions associated with otic infection. The prevention of infection includes pre-operative treatment prior to surgery as well as other suspected infectious conditions or contact. Examples of prophylaxis situations include treatment prior to surgical procedures and other operative procedures involving ear trauma or ear damage.

The topical otic compositions of the present invention may be used to treat or prevent otic infections caused by a variety of bacteria or parasites, including but not limited to one or more of the following organisms: staphylococcus aureus, proteus mirabilias, and pseudomonas aeriginosa.

The topical otic composition of the present invention may be applied to the surface of the ear, in an composition acceptable to the ear. The topical otic compositions may comprise an otically acceptable carrier and the azalide antibiotic. The “otically acceptable carrier” is used in a broad sense and includes any material or composition that can contain and release the azalide antibiotic and which is compatible with the ear. Typically the otically acceptable carrier is water or an aqueous solution or suspension, but also includes oils and polymer matrices.

The topical otic composition does not contain constituents that are physiologically or otically harmful to the ear.

Generally, for any of the more particular compositions and methods discussed herein, the amount of azalide in the topical otic composition may be in the range of from 0.001 to 10%. Preferable amounts are in the ranges of from 0.01 to 5% and 0.01 to 2%. Particular amounts are about 1-2%.

Generally, azalide antibiotics are poorly soluble in water. However, water solubility is improved if converted to a salt form. For example, azithromycin dihydrochloride has good water solubility. Accordingly, an aqueous solution of an azalide antibiotic can be formed and used for topical application to otic tissue. But, more typically, an aqueous suspension is formed of the poorly soluble or insoluble azalide antibiotic. Ointments and solid dosage forms can also be used as delivery compositions as are known in the art. The concentration of azalide antibiotic present in the otic composition depends upon the dosage form, the release rate, the dosing regimen, and the location and type of infection. Generally speaking, the concentration is from about 0.01 to 5%, more typically 0.1 to 2%, for fluid compositions and 0.5 to 50% for solid dosage forms, however, the compositions are not limited thereto.

The fluid topical otic compositions of the present invention, including both ointments and suspensions, have a viscosity that is suited for the selected route of administration. A viscosity in the range of from about 1,000 to 30,000 centipoise is useful for a drop. About 30,000 to about 100,000 centipoise is an advantageous viscosity range for otic administration in viscous solution or ribbon form. The viscosity can be controlled in many ways known to the worker skilled in the art.

The topical otic compositions may contain one or more of the following: surfactants, adjuvants including additional medicaments, buffers, antioxidants, tonicity adjusters, preservatives, thickeners or viscosity modifiers, and the like. Additives in the formulation may include sodium chloride, EDTA (disodium edetate), and/or BAK (benzalkonium chloride), sorbic acid, methyl paraben, propyl paraben, chlorhexidine, and sodium perborate. Suitable preservatives also include: polyquaternium-1, thimerosal, chlorobutanol, methyl paraben, propyl parabenl, phenylethyl alcohol, sorbic acid, or other agents known to those skilled in the art. Typically such preservatives are employed at a level of from 0.001% to 1.0% by weight.

Preservatives may be used to inhibit microbial contamination of the product when it is dispensed in single or multidose containers, and can include: quaternary ammonium derivatives, (benzalkonium chloride, benzylammonium chloride, cetylmethyl ammonium bromide, cetylpyridinium chloride), benzethonium chloride, organomercury compounds (Thimerosal, phenylmercury acetate, phenylmercury nitrate), methyl and propyl p-hydroxy-benzoates and salts thereof, betaphenylethyl alcohol, benzyl alcohol, phenylethyl alcohol and phenoxyethanol and mixtures of preservatives. These compounds are used at effective concentrations, typically from about 0.005% to about 5.0%, depending on the preservative(s) selected. The amount of the preservative used should be enough so that the solution is physically stable, i.e. a precipitate is not formed, and antibacterially effective.

A formulation in accordance with the present invention may be physically stable, that is to say no precipitate will form over the shelf life of the formulation, that an effective and potent concentration of the active ingredients will remain at the end of the shelf-life.

The solubility of the components of the present compositions may be enhanced by a surfactant or other appropriate co-solvent in the composition or solubility enhancing agents like cyclodextrins such as hydroxypropyl, hydroxyethyl, glucosyl, maltosyl and maltotriosyl derivatives of alpha-, beta-, and gamma-cyclodextrin. A particularly preferred solubility enhancer is hydroxypropyl-beta cyclodextrin (HPBC). In one embodiment, the composition comprises 0.1% to 20% hydroxypropyl-beta-cyclodextrin, more preferably 1% to 15% hydroxypropyl-beta-cyclodextrin, and even more preferably from 2.5% to 10% hydroxypropyl-beta-cyclodextrin. Co-solvents include polysorbates (for example, polysorbate 20, 60, and 80), polyoxyethylene/polyoxypropylene surfactants (e.g., Pluronic F-68, F 84 and P-103), cyclodextrin, fatty-acid glycerol-polyethylene glycol esters, other solubility agents such as Octoxynol 40, Tyloxapol and Pluronics, or other agents known to those skilled in the art and mixtures thereof. The amount of solubility enhancer used will depend on the amount of azalide antibiotic in the composition, with more solubility enhancer used for greater amounts of azalides. Typically solubility enhancers are employed at a level of from 0.01% to 20% by weight depending on the ingredient. Preferable ranges are 1% to 5% and 0.1% to 2%. Wetting agents include polyvinyl pyrolidone, polyvinyl alcholol, polyethylene glycol. The solubility agents may help keep the other components of the topical otic composition in solution, including the azalide antibiotic in solution. The wetting agent helps the formulation to spread into the ear canal.

The use of viscosity enhancing agents to provide the compositions of the invention with viscosities greater than the viscosity of simple aqueous solutions may be desirable to increase absorption of the active compounds by the target tissues or to increase the retention time in the ear. Such viscosity enhancing agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 10% by weight.

A further aspect of the present invention involves the above-mentioned use of additional medicaments in combination with the azalide antibiotic. A composition comprising an azalide antibiotic, an additional medicament, and an otically acceptable carrier can advantageously simplify administration and allow for treating or preventing multiple conditions or symptoms simultaneously. The “additional medicaments,” which can be present in any of the otic compositional forms described herein including fluid and solid forms, are pharmaceutically active compounds having efficacy in otic applications and which are compatible with an azalide antibiotic and with the ear. Typically, the additional medicaments include other antibiotics (an antibiotic that is different than an azalide antibiotic), antivirals, antifungals, anesthetics, anti-inflammatory agents, including steroidal and non-steroidal anti-inflammatories, and anti-allergic agents.

Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; comolyn; lodoxamide; levocabastin; naphazoling; antazoline; and pheniramimane. These other medicaments are generally present in a pharmaceutically effective amount as is understood by workers of ordinary skill in the art. These amounts are generally within the range of from about 0.01 to 5%, more typically 0.1 to 2%, for fluid compositions and from 0.5 to 50% for solid dosage forms.

Otic infections are frequently accompanied by inflammation of the infected otic or surrounding tissues. Similarly, otic surgical procedures that create a risk of microbial infections frequently also cause inflammation of the affected tissues. The present invention includes topical otic compositions that combine the anti-infective activity of one or more antibiotics with the anti-inflammatory activity of one or more steroid or non-steroid agents in a single composition.

The steroidal anti-inflammatory agents of the present invention include glucocorticoids, such as dexamethasone, loteprednol, rimexolone, prednisolone, Prednisolone acetate, fluorometholone, and hydrocortisone.

Dexamethasone derivatives such as U.S. Pat. No. 5,223,493, herein incorporated by reference, may also be used. Particular compounds include “21-ether derivatives of dexamethasone”, such as a 21-benzyl ether derivatives of dexamethasone.”

The preferred non-steroidal anti-inflammatory agents are: diclofenac, flurbiprofen, ketorolac, and suprofen. Other non-steroidal anti-inflammatory agents useable in the present invention include: prostaglandin H synthetase inhibitors (Cox I or Cox II), also referred to as cyclooxygenase type I and type II inhibitors, such as nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor; or other anti-inflammatory agents known to those skilled in the art.

The concentrations of the anti-inflammatory agents contained in the compositions of the present invention will vary based on the agent or agents selected and the type of inflammation being treated. The concentrations will be sufficient to reduce inflammation in the targeted otic tissues following topical application of the compositions to those tissues. Such an amount is referred to herein as “an anti-inflammatory effective amount”. The compositions of the present invention may contain one or more anti-inflammatory agents in an amount of from about 0.01 to about 5% or in a range of from about 0.1 to about 2%, as discussed above for the additional medicaments, or in a range of from about 0.01 to about 1.0 wt. %.

The aqueous otic compositions (solutions or suspensions) for use in the present invention use water and have no physiologically or otically harmful constituents. Typically purified or deionized water is used. The pH is adjusted by adding any physiologically and otically acceptable pH adjusting acids, bases or buffers to within the range of about 5.0 to 8.5. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases. pH buffers are introduced into the product to maintain a stable pH and to improve product tolerance by the user. The pH range should be 5.5-7.5.

The osmotic pressure (II) of the aqueous otic composition is generally from about 10 milliosmolar (mOsM) to about 400 mOsM, more preferably from 200-400 mOsM. If necessary, the osmotic pressure can be adjusted by using appropriate amounts of physiologically and otically acceptable salts or excipients. Sodium chloride is preferred to approximate physiologic fluid, and amounts of sodium chloride ranging from about 0.01% to about 1% by weight, and preferably from about 0.05% to about 0.45% by weight, based on the total weight of the composition, are typically used. Equivalent amounts of one or more salts made up of cations such as potassium, ammonium, sodium and the like and anions such as chloride, citrate, ascorbate, borate, phosphate, bicarbonate, sulfate, thiosulfate, and bisulfate, such as sodium bisulfate, ammonium sulfate, and the like can also be used in addition to or instead of sodium chloride to achieve osmolalities within the above-stated range. Similarly, a sugar such as mannitol, dextrose, sorbitol, glucose and the like or glycerol can also be used to adjust osmolality.

The topical otic composition of the present invention should achieve a sufficiently high tissue concentration with a minimum of doses so that a simple dosing regimen can be used to treat or prevent bacterial or parasitic infections. To this end, a preferred technique involves forming or supplying a depot of azalide antibiotic in contact with the surfaces of the ear. A depot refers to a source of azalide antibiotic that is not rapidly removed by the ear clearance mechanisms. This allows for continued, sustained high concentrations of azalide antibiotic to be present in the fluid on the surfaces of the ear by a single application. In general, it is believed that absorption are dependent on both the dissolved drug concentration and the contact duration of the external tissue with the drug-containing fluid. As the drug is removed by clearance of the fluid and/or absorption into the ear tissue, more drug is provided, e.g. dissolved, into the replenished fluid from the depot.

Accordingly, the use of a depot more easily facilitates loading of the otic tissue in view of the typically slow and low penetration rate of the generally water-insoluble/poorly soluble azalide antibiotics. The depot can effectively slowly “pump” the azalide antibiotic into the otic tissue. As the azalide antibiotic penetrates the otic tissue it is accumulated therein and not readily removed due to its long half-life. As more azalide antibiotic is “pumped” in, the tissue concentration increases and the minimum inhibit ory concentration threshold is eventually reached and/or exceeded, thereby loading the otic tissue with azalide antibiotic. By significantly exceeding the MIC₅₀, more preferably the MIC₉₀ level, provided the toxicity limit is not exceeded, a therapeutically effective concentration will remain active in the tissue for an extended period of time due to the low clearance rate of the azalide antibiotic from the tissue. Thus, depending on the depot, one or two applications may provide a complete dosing regimen. Indeed, such a simple dosing regimen may provide a 6 to 14 day treatment concentration within the otic tissue. A preferred dosing regimen involves one to two doses per day over a one to three day period, more preferably one or two doses in a single day, to provide in vivo at least a 3 day treatment and more typically a 7 day treatment.

A depot can take a variety of forms so long as the azalide antibiotic can be provided in sufficient concentration levels therein and is releasable therefrom and that the depot is not readily removed from the ear. A depot generally remains for at least about 30 minutes after administration, preferably at least 2 hours and more preferably at least 4 hours. The term “remains” means that neither the depot composition nor the azalide antibiotic is exhausted or cleared from the surface of the ear prior to the indicated time. In some embodiments, the depot can remain for up to eight hours or more. Typical otic depot forms include aqueous polymeric suspensions, ointments, and solid inserts. Polymeric suspensions are the most preferred form for the present invention and will be discussed subsequently.

The topical otic composition may be in the form of an oil. The oil solution or suspension may be a petroleum and/or silicon base to which is added the active ingredient, such as 0.1 to 2%, and excipients. Bases include mineral oil and silicon oil combinations thereof, but oil bases are not limited thereto. Since azalide antibiotics are frequently only sparingly soluble in water, an oil is an acceptable form of administration. An oil is usually applied as an ear drop. The disadvantage of oils is that they are, are messy, and may be uncomfortable/inconvenient to the patient.

An insert may be another topical otic dosage form of the present invention. Inserts are comprised of a matrix containing the active ingredient. The matrix is typically a polymer and the active ingredient is generally dispersed therein or bonded to the polymer matrix. The active ingredient is slowly released from the matrix through dissolution or hydrolysis of the covalent bond, etc. In some embodiments, the polymer is bioerodible (soluble) and the dissolution rate thereof can control the release rate of the active ingredient dispersed therein. In another form, the polymer matrix is a biodegradable polymer that breaks down such as by hydrolysis to thereby release the active ingredient bonded thereto or dispersed therein. The matrix and active ingredient can be surrounded with a polymeric coating such as in the sandwich structure of matrix/matrix+active/matrix, to further control release as is well known in the art. The kinds of polymers suitable for use as a matrix are well known in the art. The azalide antibiotic can be dispersed into the matrix material or dispersed amongst the monomer composition used to make the matrix material prior to polymerization. The amount of azalide antibiotic is generally from about 0.1 to 50%, more typically about 2 to 20%. The insert can be placed, depending on the location and the mechanism used to hold the insert in position, by either the patient or the doctor and is generally located in the ear canal. A variety of shapes and anchoring configurations, if any, are well known in the art. Preferably a biodegradable or bioerodible polymer matrix is used so that the spent insert does not have to be removed. As the biodegradable or bioerodible polymer is degraded or dissolved, the trapped azalide antibiotic is released. Although inserts can provide long term release and hence only a single application of the insert may be necessary, they are generally difficult to insert and are uncomfortable to the patient.

A preferred form of the topical otic composition of the present invention is an aqueous polymeric suspension. Here, at least one of the azalide antibiotic or the polymeric suspending agent is suspended in an aqueous medium having the properties as described above. Typically the azalide antibiotic is in suspension although it is possible for the azalide antibiotic to be in solution (water soluble) or both in solution and in suspension in significant amounts generally no less than 5% in either phase (weak to moderate water solubility and relatively high total concentrations). The polymeric suspending agent is preferably a suspension (i.e. water insoluble and/or water swellable), although water soluble suspending agents are also suitable for use with a suspension of the azalide antibiotic. The suspending agent serves to provide stability to the suspension and to increase the residence time of the dosage form in the ear. It can also enhance the sustained release of the drug in terms of both longer release times and a more uniform release curve A wetting agent is also added to improve spreading in the canal.

Examples of polymeric suspending agents include dextrans, polyethylene glycols, polyvinylpyrolidone, polysaccharide gels, Gelrite®, cellulosic polymers like hydroxypropyl methylcellulose, and carboxy-containing polymers such as polymers or copolymers of acrylic acid, as well as other polymeric demulcents. A preferred polymeric suspending agent is a water swellable, water insoluble polymer, especially a crosslinked carboxy-containing polymer.

Crosslinked carboxy-containing polymers used in practicing this invention are, in general, known in the art. In a preferred embodiment such polymers may be prepared from at least about 90% and preferably from about 95% to about 99.9% by weight, based on the total weight of monomers present, of one or more carboxy-containing monoethylenically unsaturated monomers (also occasionally referred to herein as carboxy-vinyl polymers). Acrylic acid is the preferred carboxy-containing monoethylenically unsaturated monomer, but other unsaturated, polymerizable carboxy-containing monomers, such as methacrylic acid, ethacrylic acid, .-methylacrylic acid (crotonic acid), cis-.-methylcrotonic acid (angelic acid), trans-.-methylcrotonic acid (tiglic acid), .-butylcrotonic acid, .-phenylacrylic acid, .-benzylacrylic acid, .-cyclohexylacrylic acid, .-phenylacrylic acid (cinnamic acid), coumaric acid (o-hydroxycinnamic acid), umbellic acid (p-hydroxycoumaric acid), and the like can be used in addition to or instead of acrylic acid.

Such polymers may be crosslinked by a polyfunctional crosslinking agent, preferably a difunctional crosslinking agent. The amount of crosslinking should be sufficient to form insoluble polymer particles, but not so great as to unduly interfere with sustained release of the azalide antibiotic. Typically the polymers are only lightly crosslinked. Preferably the crosslinking agent is contained in an amount of from about 0.01% to about 5%, preferably from about 0.1% to about 5.0%, and more preferably from about 0.2% to about 1%, based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, preferably alkenyl ether groupings containing terminal H₂C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053, the entire contents of which are incorporated herein by reference. Diolefinic non-hydrophilic macromeric crosslinking agents having molecular weights of from about 400 to about 8,000, such as insoluble di-acrylates and polyacrylates and methacrylates of diols and polyols, diisocyanate-hydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. Nos. 4,192,827 and 4,136,250, the entire contents of each Patent being incorporated herein by reference.

The crosslinked carboxy-vinyl polymers may be made from a carboxy-vinyl monomer or monomers as the sole monoethylenically unsaturated monomer present, together with a crosslinking agent or agents. Preferably the polymers are ones in which up to about 40%, and preferably from about 0% to about 20% by weight, of the carboxy-containing monoethylenically unsaturated monomer or monomers has been replaced by one or more non-carboxyl-containing monoethylenically unsaturated monomer or monomers containing only physiologically and otically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3hydroxypropylacrylate, and the like, vinyl acetate, N-vinylpyrrolidone, and the like; see Mueller et al. U.S. Pat. No. 4,548,990, the entire contents of which are incorporated herein by reference, for a more extensive listing of such additional monoethylenically unsaturated monomers.

Particularly preferred polymers are lightly crosslinked acrylic acid polymers wherein the crosslinking monomer is 2,3-dihydroxyhexa-1,5-diene or 2,3-dimethylhexa-1,5-diene. Preferred commercially available polymers include polycarbophil (Noveon AA-1) and Carbopol®. Most preferably, a carboxy-containing polymer system known by the tradename DuraSite®, containing polycarbophil, which is a sustained release delivery system that releases the drug at a controlled rate, is used in the aqueous polymeric suspension composition of the present invention.

The crosslinked carboxy-vinyl polymers used in practicing this invention are preferably prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, and preferably from about 3 to about 20 μm, in equivalent spherical diameter. Using polymer particles that were obtained by mechanically milling larger polymer particles to this size is preferably avoided. In general, such polymers will have a molecular weight which has been variously reported as being from about 250,000 to about 4,000,000, and from 3,000,000,000 to 4,000,000,000.

In the most preferred embodiment of the invention, the particles of crosslinked carboxy-vinyl polymer are monodisperse, meaning that they have a particle size distribution such that at least 80% of the particles fall within a 10 μm band of major particle size distribution. More preferably, at least 90% and most preferably at least 95%, of the particles fall within a 10 μm band of major particle size distribution. Also, a monodisperse particle size means that there is no more than 20%, preferably no more than 10%, and most preferably no more than 5% particles of a size below 1 μm. The use of a monodispersion of particles will give maximum viscosity and an increased ear residence time of the otic medicament delivery system for a given particle size. Monodisperse particles having a particle size of 30 μm and below are most preferred. Good particle packing is aided by a narrow particle size distribution.

The aqueous polymeric suspension normally contains 0.05 to 5%, preferably 0.5 to 2.0%, more preferably 0.5 to 1.0%, of the azalide antibiotic and 0.1 to 10%, preferably 0.5 to 6.5% of a polymeric suspending agent. In the case of the above described water insoluble, water-swellable crosslinked carboxy-vinyl polymer, a more preferred amount of the polymeric suspending agent is an amount ranging from 0.5 to 2.0%, preferably from 0.5% to about 1.2%, and in certain embodiments from 0.6 to 0.9%, based on the weight of the composition. Although referred to in the singular, it should be understood that one or more species of polymeric suspending agent such as the crosslinked carboxy-containing polymer can be used with the total amount falling within the stated ranges. In one preferred embodiment, the composition contains 0.6 to 0.8% of a polycarbophil such as NOVEON AA-1.

In one embodiment, the amount of insoluble lightly crosslinked carboxy-vinyl polymer particles, the pH, and the osmotic pressure can be correlated with each other and with the degree of crosslinking to give a composition having a viscosity in the range of from about 500 to about 100,000 centipoise, and preferably from about 1,000 to about 30,000 or about 1,000 to about 10,000 centipoise, as measured at room temperature (about 25° C.) using a Brookfield Digital LVT Viscometer equipped with a number 25 spindle and a 13R small sample adapter at 12 rpm. Alternatively, when the viscosity is within the range of 500 to 3000 centipoise, it may be determined by a Brookfield Model DV-11+, choosing a number cp-52 spindle at 6 rpm.

When water soluble polymers are used as the suspending agent, such as hydroxypropyl methylcellulose, the viscosity will typically be about 10 to about 400 centipoise, more typically about 10 to about 200 centipoises or about 10 to about 25 centipoise.

Aqueous polymeric suspensions of the present invention may be formulated so that they retain the same or substantially the same viscosity in the ear that they had prior to administration to the ear. The azalide antibiotic is released slowly as the suspended particles dissolve over time. All these events eventually lead to increased patient comfort and increased azalide antibiotic contact time with the ear tissues, thereby increasing the extent of drug absorption and duration of action of the formulation in the ear.

The viscous gels typically have residence times in the ear ranging from about 2 to about 12 hours, e.g., from about 3 to about 6 hours. The agents contained in these drug delivery systems will be released from the gels at rates that depend on such factors as the drug itself and its physical form, the extent of drug loading and the pH of the system, as well as on any drug delivery adjuvants, such as ion exchange resins compatible with the otic surface, which may also be present.

The azalide antibiotic-containing composition is topically applied to an ear of a human or non-human animal, the latter including veternary practice, such as cows, sheep, horses, pigs, goats, rabbits, dogs, cats, and other mammals. The composition can be applied as a liquid drop, ointment, a viscous solution or gel, a ribbon or as a solid. The composition can be topically applied, without limitation, to the ear canal. The application can be as a treatment of an infection in the ear or as a preventive such as prior to surgery.

All of the percentages recited herein refer to weight percent, unless otherwise indicated. The following non-limiting examples serve to illustrate certain features of the present invention.

### EXAMPLES

### Examples 1-2

Hydroxypropylmethyl cellulose, sodium chloride, edetate sodium (EDTA), BAK and surfactant are dissolved in a beaker containing approximately ⅓ of the final weight of water and stirred for 10 minutes with an overhead stirred. The azithromycin is added and stirred to disperse for 30 minutes. The solution is sterilized by autoclaving at 121° C. for 20 minutes. Alternately, the azithromycin may be dry heat sterilized and added by aseptic powder addition after sterilization. Mannitol, Poloxamer 407, and boric acid are dissolved separately in approximately ½ of the final weight of water and added by sterile filtration (0.22 μm filter) and stirred for 10 minutes to form a mixture. The mixture is adjusted to desired pH with 10N sodium hydroxide while stirring, brought to a final weight with water by sterile filtration and aseptically filled into multi-dose containers.

### Examples 3-6

Noveon AA-1 is slowly dispersed into a beaker containing approximately ⅓ of the final weight of water and stirred for 1.5 hrs. with an overhead stirrer. Noveon AA-1 is an acrylic acid polymer available from B.F. Goodrich. Edetate sodium (EDTA), BAK, sodium chloride, and surfactant are then added to the polymer solution and stirred for 10 minutes after each addition. The polymer suspension is at a pH of about 3.0-3.5. The azithromycin is added and stirred to disperse for 30 minutes. The mixture is sterilized by autoclaving at 121° C. for 20 minutes. Alternately, the azithromycin may be dry heat sterilized and added by aseptic powder addition after sterilization. Mannitol, and boric acid, or sodium perborate, Dequest, mannitol, and boric acid are dissolved separately in approximately ½ of the final weight of water, added to the polymer mixture by sterile filtration (0.22 μm filter) and stirred for 10 minutes. The mixture is adjusted to the desired pH with 10N sodium hydroxide while stirring, brought to final weight with water by sterile filtration and aseptically filled into multi-dose containers.

### Example 7

Noveon AA-1 is slowly dispersed into a beaked containing approximately ½ of the final weight of water and stirred for 1.5 hrs. With overhead stirrer. Noveon AA-1 is an acrylic acid polymer available from B.F. Goodrich. Edetate sodium (EDTA), Poloxamer 407, and sodium chloride are then added to the polymer suspension and stirred for 10 minutes. The polymer suspension is at a pH of about 3.0-3.5. The azithromycin is added and stirred to disperse for 30 minutes. The mixture is sterilized by autoclaving at 121° C. for 20 minutes. Alternately, the azithromycin may be dry heat sterilized and added by aseptic powder addition after sterilization. Mannitol is dissolved in 1/10 of the final weight of water and sterile filtered (0.22 μm filter) in to the polymer suspension and stirred for 10 minutes. The mixture is adjusted to desired pH with 10N sodium hydroxide while stirring, brought to final weight with water by sterile filtration and aseptically filled into unit-dose containers.

TABLE 1
Formulation Examples 1-7

| | 1 | 2 | 3 | 4 | 5 | 6 | 7 |
|---|---|---|---|---|---|---|---|
| Ingredient | % | % | % | % | % | % | % |
| Azithromycin | 0.10 | 0.50 | 0.10 | 0.50 | 1.0 | 1.0 | 0.10 |
| Hydroxypropyl | 0.50 | 2.00 | — | — | — | — | — |
| Cellulose | | | | | | | |
| Noveon AA-1 | — | — | 0.80 | 0.80 | 0.80 | 0.80 | 0.80 |
| Sodium Chloride | 0.20 | 0.20 | 0.20 | 0.20 | 0.20 | 0.20 | 0.30 |
| Mannitol | 1.50 | 1.50 | 1.50 | 1.50 | 1.50 | 1.50 | 1.50 |
| Edetate Disodium | 0.10 | 0.10 | 0.10 | 0.10 | | 0.10 | 0.10 |
| Poloxamer 407 | 0.10 | 0.10 | 0.10 | 0.10 | 0.10 | 0.10 | 0.10 |
| Benzalkonium | 0.01 | 0.01 | 0.01 | 0.01 | — | 0.01 | — |
| Chloride | | | | | | | |
| Sodium Perborate | — | — | — | — | 0.10 | — | — |
| Dequest 2060S | — | — | — | — | 0.10 | — | — |
| Boric Acid | — | — | — | — | 0.50 | — | — |
| Citric Acid | .2 | .2 | .2 | .2 | — | .2 | .2 |
| Sodium Citrate | .17 | .17 | .17 | .17 | — | .17 | .17 |
| Sodium Hydroxide | q.s. to | q.s. to | q.s. to | q.s. to | q.s. to | q.s. to | q.s. to |
| | pH 6.2 | pH 7 | pH 6.3 | pH 6.3 | pH 6.3 | pH 6.3 | pH 6 |
| Water | q.s. | q.s. | q.s. | q.s. | q.s. | q.s. | q.s. |
| | to | to | to | to | to | to | to |
| | 100 | 100 | 100 | 100 | 100 | 100 | 100 |

### Example 8

An azithromycin ointment is prepared by dissolving 0.3 grams of azithromycin and 0.5 grams of chlorobutanol in a mixture containing 3.0 grams mineral oil/96.2 grams white petrolatum by stirring in a 100 ml beaker while heating sufficiently hot to dissolve both compounds. The mixture is sterile filtered through a 0.22 μm filter at a sufficient temperature to be filtered and filled aseptically into sterile ophthalmic ointment tubes.

### Examples 9-11

Hydroxypropylmethyl cellulose (HPMC), sodium chloride, edetate sodium (EDTA), and surfactant are dissolved in a beaker containing approximately ⅓ of the final weight of water and stirred for 10 minutes with an overhead stirrer. The mixture is sterilized by autoclaving at 121° C., for 20 minutes. The azithromycin and steroid as indicated in table 2 are dry heat sterilized and added to the HPMC-containing solution by aseptic powder addition. Mannitol, Poloxamer 407, BAK, and boric acid are dissolved separately in approximately ½ of the final weight of water and added by sterile filtration (0.22 μm filter) and stirred for 10 minutes to form a mixture. The mixture is adjusted to desired pH with 10N sodium hydroxide while stirring, brought to a final weight with water by sterile filtration, and aseptically filled into multi-dose containers.

### Examples 12-14

Noveon AA-1 is slowly dispersed into a beaker containing approximately ⅓ of the final weight of water and stirred for 1.5 hrs. with an overhead stirrer. Noveon AA-1 is an acrylic acid polymer available from B.F. Goodrich. Edetate sodium (EDTA), sodium chloride, and surfactant are then added to the polymer solution and stirred for 10 minutes after each addition. The polymer suspension is at a pH of about 3.0-3.5. The mixture is sterilized by autoclaving at 121° C. for 20 minutes. The azithromycin and steroid as indicated in table 2 are dry heat sterilized and added to the polymer suspension by aseptic powder addition. BAK, mannitol, and boric acid are dissolved separately in approximately ½ of the final weight of water, added to the polymer mixture by sterile filtration (0.22 μm filter) and stirred for 10 minutes. The mixture is adjusted to the desired pH with 10N sodium hydroxide while stirring, brought to final weight with water and by sterile filtration and aseptically filled into multi-dose containers.

TABLE 2
Formulation Examples 9-14

| | 9 | 10 | 11 | 12 | 13 | 14 |
|---|---|---|---|---|---|---|
| Ingredient | % | % | % | % | % | % |
| Azithromycin | 1.0 | 1.0 | 1.0 | 2.0 | 2.0 | 2.0 |
| Prednisoline Acetate | 0.10 | — | — | 0.10 | — | — |
| Fluorometholone | — | 0.10 | — | — | 0.10 | — |
| Dexamethasone | — | — | 0.10 | — | — | 0.10 |
| Hydroxypropyl methyl | 1.50 | 1.50 | 1.50 | — | — | — |
| Cellulose | | | | | | |
| Noveon AA-1 | — | — | — | 0.80 | 0.80 | 0.80 |
| Sodium Chloride | 0.20 | 0.20 | 0.20 | 0.20 | 0.20 | 0.20 |
| Mannitol | 1.50 | 1.50 | 1.50 | 1.50 | 1.50 | 1.50 |
| Edetate Disodium | 0.10 | 0.10 | 0.10 | 0.10 | 0.10 | 0.10 |
| Poloxamer 407 | 0.10 | 0.10 | 0.10 | 0.10 | 0.50 | 0.50 |
| Benzalkonium | 0.01 | 0.01 | 0.01 | 0.01 | 0.01 | 0.01 |
| Chloride | | | | | | |
| Boric Acid | 0.50 | 0.50 | 0.50 | 0.50 | 0.50 | 0.50 |
| Sodium Hydroxide | q.s. | q.s. | q.s. | q.s. | q.s. | q.s. |
| | to | to | to | to | to | to |
| | pH 7 | pH 7 | pH 7 | pH 6 | pH 6 | pH 6 |
| Water | q.s. | q.s. | q.s. | q.s. | q.s. | q.s. |
| | to | to | to | to | to | to |
| | 100 | 100 | 100 | 100 | 100 | 100 |

### Examples 15 and 16

The azithromycin is dissolved in a citrate buffer. After dissolving, the mannitol, EDTA, and BAC are added and dissolved in the azithromycin solution. The volume of the formulation is adjusted to 100% of the desired volume with water. 2N sodium hydroxide is used to adjust the pH. The solution is then filtered through a 0.22 um filter to produce a sterile solution. The azithromycin used here can be the free base, monohydrate or dihydrate forms.

TABLE 3

| | Example 15 | Example 16 |
|---|---|---|
| Ingredient | % | % |
| % Azithromycin | 1.0 | 2.0 |
| Sodium Citrate | 0.20 | 0.3 |
| Citric Acid | 0.14 | 0.14 |
| Mannitol | 4.6 | 4.5 |
| EDTA | 0.05 | 0.05 |
| Benzalkonium Chloride (BAC) | 0.005 | 0.005 |
| Water | qs to 100 | qs to 100 |
| Sodium Hydroxide | qs to pH 7.0 | qs to pH 7.0 |

### Examples 17-23

Additional Formulations.

TABLE 4
Formulation Examples 17-23

| | 17 | 18 | 19 | 20 | 21 | 22 | 23 |
|---|---|---|---|---|---|---|---|
| Ingredient | % | % | % | % | % | % | % |
| Azithromycin | 01.0 | 2.0 | 1.0 | 2.0 | 1.0 | 2.0 | 0.10 |
| Hydroxypropyl | 0.50 | 2.00 | — | — | — | — | — |
| Cellulose | | | | | | | |
| Noveon AA-1 | — | — | 0.80 | 1.30 | 0.80 | 0.80 | 0.80 |
| Sodium Chloride | 0.20 | 0.20 | 0.20 | 0.20 | 0.20 | 0.20 | 0.30 |
| Mannitol | 1.50 | 1.50 | 1.50 | 1.50 | 1.50 | 1.50 | 1.50 |
| Edetate Disodium | 0.10 | 0.10 | 0.10 | 0.10 | | 0.10 | 0.10 |
| Poloxamer 407 | 0.10 | 0.10 | 0.10 | 0.10 | 0.50 | 0.50 | 0.50 |
| Benzalkonium | 0.01 | 0.01 | 0.01 | 0.01 | — | 0.01 | — |
| Polyvinyl pyrolidone Chloride | | | | 0.2 | | | |
| Sodium Perborate | — | — | — | — | 0.10 | — | — |
| Dequest 2060S | — | — | — | — | 0.10 | — | — |
| Boric Acid | | | | | 0.50 | | |
| Citric Acid | .2 | .25 | .2 | .25 | | .25 | .2 |
| Sodium Citrate | .17 | .17 | .17 | .17 | | .17 | .17 |
| Sodium Hydroxide | q.s. | q.s. | q.s. | q.s. | q.s. | q.s. | q.s. |
| | to | to | to | to | to | to | to |
| | pH 6.3 | pH 7 | pH 6.3 | pH 6.3 | pH 6.3 | pH 6.3 | pH 6 |
| Water | q.s. | q.s. | q.s. | q.s. | q.s. | q.s. | q.s. |
| | to | to | to | to | to | to | to |
| | 100 | 100 | 100 | 100 | 100 | 100 | 100 |

The above discussion of this invention is directed primarily to preferred embodiments and practices thereof. It will be readily apparent to those skilled in the art that further changes and modifications in actual implementation of the concepts described herein can easily be made or may be learned by practice of the invention, without departing from the spirit and scope of the invention as defined by the following claims.

We claim:

1. A topical otic composition comprising an azalide antibiotic and an otically acceptable carrier.

2: The topical otic composition according to claim 1, wherein said topical otic composition comprises an additional medicament which is different than an azalide antibiotic.

3: The topical otic composition according to claim 2, wherein said additional medicament is selected from the group consisting of antibiotics, antivirals, antifungals, anesthetics, anti-inflammatory agents, and anti-allergic agents.

4: The topical otic composition according to claim 2, wherein said topical otic composition further comprises a polymeric suspending agent or a wetting agent.

5. The topical otic composition according to claim 2, wherein said additional medicament is a steroidal anti-inflammatory agent.

6: The topical otic composition according to claim 5, wherein said steroidal anti-inflammatory agent is selected from the group consisting of Prednisolone acetate, Fluorometholone, Dexamethasone and a pharmaceutically acceptable salt thereof.

7. The topical otic composition according to claim 2, wherein said additional medicament is a non-steroidal anti-inflammatory agent.

8: The process for treating an ear of claim 7, wherein said non-steroidal anti-inflammatory agent is selected from the group consisting of diclofenac, flurbiprofen, ketorolac, and suprofen.

9: The topical otic composition according to claim 1, wherein said topical otic composition comprises a polymeric suspending agent.

10: The topical otic composition according to claim 9, wherein said polymeric suspending agent is a water-swellable water-insoluble crosslinked carboxy-vinyl polymer.

11: The topical otic composition according to claim 1, wherein said azalide antibiotic is a compound of formula (I): 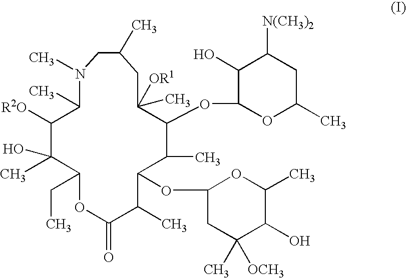
wherein R¹ and R² each independently represent a hydrogen atom or methyl group.

12: The topical otic composition according to claim 1, wherein the amount of said azalide antibiotic is at least about 5.0%.

13: The topical otic composition according to claim 1, wherein the amount of said azalide antibiotic is from about 0.01% to about 10%.

14: The topical otic composition according to claim 1, wherein the amount of said azalide antibiotic is from about 0.01% to about 2%.

15: The topical otic composition according to claim 1, wherein the amount of said azalide antibiotic is from about 1% to about 2%.

16: The topical otic composition according to claim 1, wherein said topical otic composition is in the form of a depot.

17: The topical otic composition according to claim 1, wherein said topical otic composition is in the form of an aqueous solution, aqueous suspension, an oil, or an insert.

18: The topical otic composition according to claim 1, wherein said topical otic composition does not contain constituents that are physiologically or otically harmful to the ear.

19. A process for treating an ear, which comprises:
topically applying a topical otic composition to an ear, wherein said topical otic composition comprises an azalide antibiotic in an amount effective to treat infection in a tissue of the ear and an ophthalmically acceptable carrier.

20: The process for treating an ear according to claim 19, wherein said topical otic composition comprises an additional medicament which is different than an azalide antibiotic.

21: The process for treating an ear according to claim 20, wherein said additional medicament is selected from the group consisting of antibiotics, antivirals, antifungals, anesthetics, anti-inflammatory agents, and anti-allergic agents.

22: The process for treating an ear according to claim 20, wherein said topical otic composition further comprises a polymeric suspending agent or a wetting agent.

23: The process for treating an ear according to claim 20, wherein said additional medicament is a steroidal anti-inflammatory agent.

24: The process for treating an ear according to claim 23, wherein said steroidal anti-inflammatory agent is selected from the group consisting of Prednisolone acetate, Fluorometholone, Dexamethasone and a pharmaceutically acceptable salt thereof.

25. The process for treating an ear according to claim 20, wherein said additional medicament is a non-steroidal anti-inflammatory agent.

26: The process for treating an ear according to claim 25, wherein said non-steroidal anti-inflammatory agent is selected from the group consisting of diclofenac, flurbiprofen, ketorolac, and suprofen.

27: The process for treating an ear according to claim 19, wherein said topical otic composition comprises a polymeric suspending agent.

28: The process for treating an ear according to claim 27, wherein said polymeric suspending agent is a water-swellable water-insoluble crosslinked carboxy-vinyl polymer.

29: The process for treating an ear according to claim 19, wherein said azalide antibiotic is a compound of formula (I): 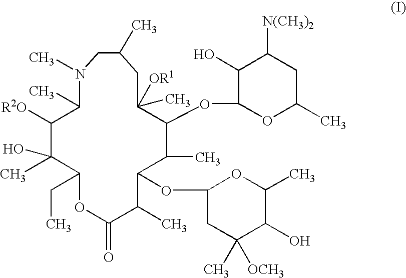
wherein R¹ and R² each independently represent a hydrogen atom or methyl group.

30: The process for treating an ear according to claim 19, wherein the amount of said azalide antibiotic is at least about 5.0%.

31: The process for treating an ear according to claim 19, wherein the amount of said azalide antibiotic is from about 0.01% to about 10%.

32: The process for treating an ear according to claim 19, wherein the amount of said azalide antibiotic is from about 0.01% to about 2%.

33: The process for treating an ear according to claim 19, wherein the amount of said azalide antibiotic is from about 1% to about 2%.

34: The process for treating an ear according to claim 19, wherein said topical otic composition is in the form of a depot.

35: The process for treating an ear according to claim 19, wherein said topical otic composition is in the form of an aqueous solution, aqueous suspension, an oil, or an insert.

36: The process for treating an ear according to claim 19, wherein said ear is suffering from at least one condition selected from the group consisting of otitis externa, otitis media, ear inflammation, ear infection and ear trauma.

37: The process for treating an ear according to claim 19, wherein said infection is caused by a bacteria selected from the group consisting of corneform and idphtheroids.

38: A process for treating an ear, which comprises:
topically applying a topical otic composition to an ear during or following an otic surgical procedure, wherein said topical otic composition comprises an azalide antibiotic and an ophthalmically acceptable carrier.

39: The process for treating an ear according to claim 38, wherein otic surgical procedure is tympanostomy.
